(19) United States
(12) Patent Application Publication
(10) Pub. No.: US 2012/0180149 A1
(43) Pub. Date: Jul. 12, 2012

(54) DOUBLE MUTANT MOUSE AND CELL LINES

(75) Inventor: Timothy A. Lyerla, Eugene, OR (US)

(73) Assignee: CLARK UNIVERSITY, Worcester, MA (US)

(21) Appl. No.: 13/496,572

(22) PCT Filed: Sep. 16, 2010
(86) PCT No.: PCT/US10/49064

§ 371 Date: Mar. 16, 2012

Related U.S. Application Data
(60) Provisional application No. 61/243,280, filed on Sep. 17, 2009.

(51) Int. Cl. A01K 67/027; C12Q 1/02; G01N 33/566; C12N 5/10

(52) U.S. Cl. 800/9; 435/354; 435/29; 435/7.21

(57) ABSTRACT

A mutant transgenic mouse and cell line derived from the mouse are disclosed. The mutant transgenic mouse was developed from a cross between a mutant mouse which carries mutant genes that express a phenotype similar to human Hermansky-Pudlak Syndrome, and a mouse strain containing a transgene encoding a temperature sensitive protein that is inactive at physiological temperatures. The resulting mutant mouse is characterized by the presence of the HPS mutations as well as the transgene. The mutant mouse and cell lines derived from the lung tissue of the mouse are useful models for lung pathology associated with human HPS, lung fibrosis and inflammation. Methods and assays utilizing the cell lines also are disclosed.

### RELATED APPLICATIONS

This application claims the benefit under 35 U.S.C. §119(e) of U.S. provisional application Ser. No. 61/243,280, filed 17 Sep. 2009, the entirety of which is incorporated herein by reference.

### BACKGROUND OF THE INVENTION

Hermansky-Pudlak Syndrome (HPS) is a rare genetically heterogeneous disorder that affects lysosome organelles, including melanosomes, platelet dense bodies and alveolar type II cells (1, 2). At least eight genetically distinct forms of HPS have been described in humans, of which HPS1, which is caused by mutations in the HPS1 gene, is the most prevalent (3). HPS causes hypopigmentation and prolonged bleeding times in afflicted persons, many of whom develop lung pathology. The most serious clinical consequence of HPS is lung fibrosis, which often leads to death in midlife (1, 4-5). Lung fibrosis is a cellular disease involving the replacement of epithelial tissue by the deposition of collagen from pulmonary fibroblasts.

HPS presently has no cure, and its molecular causes are unknown. Animal mutants that accurately model human HPS, and cell lines derived from these animals, are important for studying the biological processes involved in HPS. Several mouse hypopigmentation mutants exist that model human HPS (6-11). Animal models for HPS lung abnormalities also have been described (12, 13).

The ability to study biological function on the cellular level has greatly benefited from the availability of stable cell lines which allow biochemical experimentation to be conducted on homogenous populations of clonally derived cells. Genes which allow the generation of cell lines share the property of preventing cells from differentiating into a non-dividing end-stage cell. This family of genes has been called differentiation-inhibiting genes or immortalizing genes (14, 15, 16). The cells in which these genes are expressed can be established as tissue culture lines which will grow for effectively infinite periods in vitro.

The differentiation-inhibiting genes frequently are members of a family of oncogenes called the nuclear oncogenes. These nuclear oncogenes include a number of viral oncogenes (e.g., SV40 large T antigen, polyoma large T antigen, human papilloma virus E7 antigen) and also a number of genes with known cellular homologs (17, 18, 19). It has been shown that the simian virus 40 (SV40) large tumor antigen (TAg) by itself is capable of efficiently immortalizing primary fibroblasts (17). A variety of methods have been used to place immortalizing oncogenes into cells to allow the establishment of cell lines. For example, transfection and retroviral-mediated insertion of immortalizing genes have been reported (20-22). Another method utilizes a murine retrovirus shuttle vector system to construct recombinant retroviruses for infection of rat Fill cells (23).

In another approach, immortalizing genes have been introduced into mice to produce transgenic mice whose genome incorporates an immortalizing gene, e.g., SV40 large T antigen (24). Because many of these genes are oncogenic, transgenic animals in which the immortalizing transgene is expressed under in vitro culture conditions, but not under normal physiological conditions, have been developed (25). Cell lines derived from these animals express the immortalizing gene under the appropriate conditions, permitting an immortalized cell line to be established.

### SUMMARY OF THE INVENTION

The present invention comprises a mutant transgenic mouse that accurately models the lung pathology of human HPS, and that contains an immortalizing transgene that facilitates the establishment of stable cell lines derived from the mouse. This mutant mouse stain can be used for the production of cell lines that provide bioassays for presymptomatic conditions of lung disorders associated with HPS, including pulmonary fibrosis. The present invention includes assays and methods using the mutant mouse and cell lines.

The present mutant mouse was developed from a cross between a mutant mouse which is characterized by a mutation in at least one gene that has been associated with human HPS (“HPS mouse”), and a mouse strain containing an immortalizing transgene (“immortalizing mouse”). In a currently preferred embodiment, the HPS mouse is a double mutant that is homozygous for the pale ear (HPS1/ep) and pearl (Ap3b1/pe) genes, and the immortalizing mouse contains the temperature-sensitive SV40tsA58 transgene. The resulting double mutant mouse is characterized by the presence of the mutations as well as the SV40tsA58 transgene.

The present invention further comprises a stable cell line established from cells extracted from the present mutant transgenic mouse. The cell lines of the present invention are useful models for various pathologies associated with HPS. The double mutant transgenic mouse is a useful model for lung inflammation in the development of lung fibrosis and other pulmonary disorders associated with HPS. Methods and assays utilizing the cell lines also are disclosed.

### DETAILED DESCRIPTION OF THE INVENTION

Hermansky-Pudlak Syndrome (HPS) is a rare autosomal recessive disorder which results in oculocutaneous albinism (decreased pigmentation), bleeding problems due to a platelet abnormality (platelet storage pool defect), and occasional storage of an abnormal fat-protein compound (lysosomal accumulation of ceroid lipofuscin). The disease causes dysfunctions of the lungs, intestine, kidneys or heart. The major complication of most forms of the disorder is pulmonary fibrosis, which typically exhibits in patients between about 40-50 years old. Pulmonary fibrosis is a fatal complication in at many forms of HPS, and is the usual cause of death from the disorder. The general prognosis for persons afflicted with HPS is considered to be poor.

Stable cell lines that express a phenotype characteristic of HPS are important components in the understanding of HPS, and the clinical development of treatments for HPS. The present invention provides a mutant mouse whose cells can be used for developing established cell lines. The cell lines of the present invention can be used, for example, in bioassays for cytokines involved in the inflammatory process that are normally undetectable using other methods, such as ELISA or mass spectrometry. These cytokines would provide biomarkers for preconditions leading to pulmonary fibrosis and other lung disorders in HPS patients. Early recognition of these diseases, prior to their overt symptomatic development, may allow for therapeutic intervention in order to prevent or abrogate the disease process.

HPS can be caused by mutations in several genes, including HPS1, HPS3, HPS4, HPS5 and HPS6; HPS2, which includes immunodeficiency in its phenotype, is caused by mutation in the Ap3b1 gene. HPS7 is caused by mutation in the DTNBP1 gene, and HPS8 is caused by mutation in the BLOC1S3 gene. (See, the OMIM database, available through the NCBI website). HPS often is characterized by mutations in one or more of these genes.

A number of animal models exist having mutant phenotypes similar to human HPS. For example, an HPS1 mouse model has two genetically distinct mouse loci, “pale ear” (ep) and “ruby-eye” (ru), that map close together in the homologous region of murine chromosome 19, which suggested that one of these loci might be homologous to human HPS (26). Other animal models are listed in Table A:

TABLE A
Animal models for HPS

| HPS | Animal model | |
|---|---|---|
| Gene | phenotype | Reference |
| HPS1 | “pale ear” (ep) | Feng et al., Hum. Mol. Genet., 6: |
| | | 793-97 (1997). |
| Ap3b1 | ‘pearl” (pe) | Balkema et al., Science, 219: 1085- |
| (HPS2) | | 1087, 1983. |
| HPS3 | “cocoa” (coa) | Suzuki, et al., Genomics 78: 30-37, |
| | mouse | 2001. |
| HPS4 | ‘light ear’ (le) | Lane and Green, J. Hered. 58: 17-20, |
| | | 1967. |
| HPS5 | “ruby eye 2” | Zhang et al., Nature Genet. 33: |
| | (ru2) | 145-154, 2003 |
| HPS6 | ru | Zhang et al., Nature Genet. 33: |
| | | 145-154, 2003 |
| DTNBP1 | “sandy” (sdy) | Swank et al., Genet. Res. Camb. 58: |
| (HPS7) | | 51-62, 1991. |
| BLOC1S3 | “reduced | Starcevic and Dell'Angelica, J. Biol. |
| (HPS8) | pigmentation” (rp) | Chem. 279: 28393-28401, 2004 |

A number of other animal models for HPS exist. See, e.g., Lyerla et al., Am J Physiol—Lung Cell Mol Physiol, 285:L643-L653 (2003) and the references cited therein, and the website for the Jackson Laboratory (Bar Harbor, Me.).

The present invention comprises a mutant HPS mouse that contains at least one HPS gene that has been associated with human HPS, and an immortalizing transgene, which is obtained by mating an HPS mouse and an immortalizing mouse. The term “HPS mouse” refers to a mouse that contains at least one HPS gene. The HPS genes may be selected from the group consisting of: HPS1, HPS3, HPS4, HPS5, HPS6, Ap3b1,DTNBP1 and BLOC1S3 or a combination of two or more of these genes. In a preferred embodiment, the HPS mouse contains at least two HPS genes, and has a phenotype that emulates the lung pathology of human HPS. In a currently preferred embodiment, the HPS mouse is homozygous for the pale ear (HPS1/ep) and pearl (Ap3b1/pe) genes.

The HPS mouse is mated with a mouse containing a transgene that, when expressed in cells, immortalizes the cells, that is, prevents the cells from differentiating into a non-dividing end-stage (referred to herein as an “immortalizing mouse”). Immortalizing transgenes include, for example, genes that encode the SV40 large tumor antigen (SV40-TAg), the polyomavirus large T antigen (Py-TAg), the polyomavirus middle T antigen, the human papilloma virus E7 antigen (HPV-E7), cellular p53 protein, the adenovirus E1a protein, and the myc protein. Many immortalizing genes are oncogenic, therefore when the gene is to be incorporated into a living animal, it is preferable to utilize a form of the gene that is inactive or not expressed in the animal, but that will be expressed in cultured cells. Such genes include, for example, temperature sensitive variants that are inactive at the animal's physiological temperature, but become active at the lower temperatures in a laboratory or other environment. In a currently preferred embodiment, the transgene is the temperature-sensitive SV40tsA58 transgene.

In a currently preferred embodiment, the HPS mouse characterized by the presence of the mutations as well as the SV40tsA58 transgene. In this embodiment, the mouse is produced by crossing an HPS double mutant mouse with an immortalized mouse. The HPS double mutant preferably is C57/BL6-HPS1ep Ap3b1/pe, which is double homozygous for the pale ear (HPS1ep) and pearl (A3pb1pe) genes (the “ep/pe double mutant”), and which is an animal model for HPS lung disease (Lyerla et al., (2003), supra). Some of the hallmark features of this animal include partial albinism that also involves eye pigmentation, enlarged alveolar type II cells, and activated alveolar macrophages. Older animals develop pulmonary fibrosis when raised in an open colony, and levels of the fibrogenetic cytokine, TGF-β1, are elevated throughout post-natal life in this strain. This strain is affected with lung conditions that lead to serious pulmonary disease.

The ep/pe double mutant mice were mated with a commercially available strain: an C3H inbred mouse strain that has been transfected with the temperature sensitive SV40tsA58 transgene (Immortomouse®) to produce the transgenic mouse. When cells derived from the Immortomouse® are cultured at 33° C., the SV40tsA58 transgene is expressed and produces an active SV40 large T antigen protein that blocks cell division checkpoints to produce cells that are under continuous mitosis. i.e., an “immortalized” or established cell line. When the temperature is increased to 37° C., the transgene becomes inactive, and the cells undergo differentiation according to their cell type.

The resulting progeny then are tested to determine which contain both the ep/pe double mutation and the SV40tsA58 transgene; approximately one-eighth of the progeny contain both the double mutation and the transgene. This strain is phenotypically ep/pe by coat and eye color, and is positive for the presence of the transgene. The presence of the transgene cannot be seen phenotypically, but can be determined by conventional genetic analysis, e.g., using polymerase chain reaction (PCR) for amplification, and visualization of a portion of the gene by agarose gel electrophoresis. The immortalized double mutant mice containing both the ep/pe double mutation and the SV40tsA58 transgene exhibit the same HPS lung pathology as the parent ep/pe double mutant mice.

The present invention further comprises stable cell lines established from the cells of the immortalized double mutant mouse. Techniques for establishing cell lines from transgenic mice containing a temperature-sensitive transgene are well known. See, for example, Whitehead et al., PNAS USA, 90:587-591 (1993); Jat et al., PNAS USA, 88:5096-5100 (1991); and Whitehead and Robinson, Am J Physiol Gastrointest Living Physiol, 296:G455-G460 (2008) and the references cited therein., the entirety of which are incorporated herein by reference.

In a preferred embodiment of the present invention, cell lines are derived from alveolar macrophage cells isolated from bronchial alveoli lavage samples. Techniques for isolating cells from lavage samples and establishing a cell line from the cells have been disclosed, for example, by Lyerla et al, (2003), supra. In a preferred aspect, an alveolar macrophage line is derived from lung tissue from the present double mutant animals that have been lavaged with saline or another lavage solution. Cells are collected from the lavage fluid by centrifugation at room temperature, and the pellet is resuspended, e.g., in saline, a buffer or phospho-buffered saline (PBS). Non-lysed cells may be recovered by centrifugation, and cultured, e.g., in microwell plates for attachment and formation of monolayers in DMEM culture medium at about 33° C. The cells are cultured at a temperature appropriate for activation of the transgene (33° C.) to form a stable cell line.

In another preferred embodiment, cell lines are derived from lung epithelium cells. Techniques for isolating cells from tissue samples and establishing a cell line from the cells are known. (see, e.g., Whitehead and Robinson, (2008) Am J Physiol—Gastrointestinal and Liver Physiol., 296:G455-G460). In a preferred aspect, a lung epithelial line is derived from lung tissue from the present double mutant animals that had been digested in situ with dispase after first blanching the lung with saline and lavaging to remove free alveolar cells. The resulting cell suspension digest is panned in antibody coated plates to remove non-epithelial cells, and floating epithelial cells cultured, e.g., in microwell plates for attachment and formation of monolayers in DMEM culture medium at about 33° C. The cultures may be expanded by detachment with a protease such as trypsin, and subcultured under the same conditions to provide sufficient numbers of cells for passaging in larger cell culture vessels and freezing samples for long term storage. The cells are cultured at a temperature appropriate for activation of the transgene (33° C.) to form a stable cell line.

The double mutant mice of the present invention exhibit lung pathology that is similar to that evidenced by human HPS patients, including enlargement of type II cells and lamellar bodies, and the presence of surfactant in the lamellar bodies. In addition, the air spaces of the lungs of the mutant mice contain inflammatory cells and foamy macrophages. Moreover, the present double mutant mice exhibit early on characteristics that are indicative of lung inflammation which often precedes the development of pulmonary fibrosis; these characteristics include highly activated macrophages that develop post-natally within about the first month, and have high levels of TGF-β1 within about three months. Therefore, the present mutant mice provide a useful model for studying the pathology of human HPS and lung fibrosis, for identifying biomarkers predictive of the development lung fibrosis, and/or for determining the effectiveness of therapeutic interventions for human HPS and lung fibrosis.

The present mutant mice also are useful as a source of cells that can be used to form immortalized cell lines that contain both the HPS genes and the conditionally-expressed immortalizing gene. Cells can be derived from any organ and used to form a cell line that, cultured under the appropriate conditions, expresses the immortalizing gene. Such cell lines are useful for studying the pathology of HPS in various organs. In a currently preferred aspect, the mutant mice are ep/pe double mutant mice. Bronchial alveolar macrophage cells derived from immortalized ep/pe mutant mice exhibit many of the morphological and biochemical abnormalities evidenced by the lungs of human HPS patients.

Cell lines derived from the present double mutant mouse can be used in bioassays for diagnostic or prognostic purposes, or for identifying potential therapeutic candidates for treatment of HPS, lung fibrosis or inflammation. In a currently preferred aspect of the present invention, alveolar macrophage cells and lung epithelial cells derived from the present mouse are used to establish highly stable immortalized cell lines useful for studying various aspects of the lung pathology of HPS, including lung fibrosis and inflammation.

Candidate compounds can be tested using the cell lines of the present invention to identify those showing promise for the amelioration or treatment of HPS, lung fibrosis and/or inflammation. Such candidate compounds include those that are capable of binding, activating or modulating the activity of a cytokine or other molecule associated with HPS, lung fibrosis or inflammation, such as TGF-beta1, or compounds that modulate the activity of a Toll-like receptor (TLR) or molecule in the TLR pathway. The cell lines of the present invention are particularly useful for such use in screening assays because, unlike prior cell lines derived from HPS-model mice, the cell lines of the present invention are highly stable and do not exhibit the variability of expression that characterize many prior cell lines.

Types of assays that can be carries out utilizing the present cell lines include, for example, apoptosis assays, cell proliferation assays, cytotoxicity assays, reporter gene assays, protein assays, and other bioassays. Cells derived from the present immortalized ep/pe double mutant mouse are particularly useful for bioassays for cytokines and other molecules involved in the pathology of HPS, lung fibrosis and inflammation.

In one aspect, cells derived from the present double mutant mouse can be used in a bioassay for screening potential agonists and/or antagonists to Toll-like receptor (TLR) proteins, including TLR proteins and proteins in the TLR pathway. Both TLR4 and TLR2 receptors are expressed and functionally active on the alveolar macrophages. Cabanski et al., (2008), Am. J. Respir. Cell Mol. Biol., 48: 26-31.

In a currently preferred embodiment, cells of the present invention are used in a bioassay for screening compounds that stimulate or inhibit the activity of a TLR, particularly TLR4 or TLR2, that show promise as immunoenhancive or immunosuppressive agents. Highly sensitive assays can be performed using the present cell lines that are especially useful for identifying compounds that work at very low doses, and for identifying side effects manifesting at the cellular level resulting from administration of the compound.

Toll-like receptors (TLRs) recognize distinct pathogen-associated molecular patterns and play a critical role in innate immune responses. They participate in the first line of defense against invading pathogens and play a significant role in inflammation, immune cell regulation, survival and proliferation. Several members of the TLR family have been identified, of which TLR1, 2, 4, 5 and 6 are located on the cell surface and TLR3, 7, 8 and 9 are localized to the endosomal/lysosomal compartment. Triggering of the TLR pathway leads to the activation of NF-κB and subsequent regulation of immune and inflammatory genes. The TLRs and members of the IL-1 receptor family share a conserved stretch of approximately 200 amino acids known as the TIR domain. Upon activation, TLRs associate with a number of cytoplasmic adaptor proteins containing TIR domains including MyD88 (myeloid differentiation factor), MAL/TIRAP (MyD88-adaptor-like/TIR-associated protein), TRIF (Toll-receptor-associated activator of interferon) and TRAM (Toll-receptor-associated molecule). This association leads to the recruitment and activation of IRAK1 and IRAK4, which form a complex with TRAF6 to activate TAK1 and IKK. Activation of IKK leads to the degradation of IκB that normally maintains NF-κB inactivity by sequestering it in the cytoplasm.

A TLR agonist is generally any agent that can bind or ligate a TLR, and stimulate the activity of the TLR through downstream signaling or receptor activation. For example, stimulation of the TLR4 receptor can be determined by the ensuing downstream signal transduction, initiating an adaptive immune response. Adaptor molecules involved in TLR signaling include, for example, MyD88, Tirap (Mal), Trif and Tram. Shigeoka et al., (2007), J. Immunol., 178 (10): 6252-8; Yamamoto et al., (2003), Nat. Immunol., 4 (11): 1144-50; Yamamoto et al., (2002), Nature, 420 (6913): 324-9. The adapters activate other molecules within the cell, including certain protein kinases (IRAK1, IRAK4, TBK1 and IKKi) that amplify the signal and lead to induction or suppression of an inflammatory response.

A TLR antagonist is a compound that inhibits TLR receptor activation. TLRs are important components of pathogen recognition, and TLR stimulation may activate key signaling molecules involves in innate immune activation. Activation via TLRs leads to the production of proinflammatory cytokines, chemokines and surface molecules that play a key role in the regulation and control of inflammatory reactions and adaptive immunity. Inappropriate activation of TLRs can result in sterile inflammation or autoimmunity. For example, TLR7 and TLR9 activation by endogenous RNA and DNA, transported to the endosomes in the form of immune complexes or non-covalently associated with cationic peptides, could be an important mechanism involved in promoting diseases such as systemic lupus erythematosus, psoriasis and other autoimmune diseases. Barrat and Coffman, Immunol Rev., (2008) 223:271-83. Compounds that inhibit these receptors show promise as therapeutic agents for autoimmunity.

Screening assay utilizing cells of the present invention may be a cell-based or cell-free binding assay. In one aspect of the present invention, methods for screening for a candidate compound that modulates the activity of a TLR receptor, in particular TLR2 and/or TLR4, for the amelioration or treatment of lung fibrosis, HPS or lung inflammation is provided. The method comprises (a) contacting an alveolar macrophage cell or lung epithelial cell of the present invention with a test compound; (b) contacting the same cell used in step (a) with a diluent in the absence of the test compound to form a control cell; (c) determining that the test compound is a ligand for a TLR receptor; (d) comparing the level of interaction (e.g., agonist or antagonist activity) between the test compound and the TLR receptor in the test and control cells to identify a significant difference therein; and (e) determining based on the differences in activity found in step (d), whether the test compound is a candidate compound for the treatment or amelioration of lung fibrosis, HPS or lung inflammation. Compounds identified using the cells of the present invention as agonists or antagonists of TLR 2 and/or TLR4 have the potential for providing immunoenhancive or immunosuppressive activity.

Assays for cytokine production of an established alveolar macrophage line or lung epithelial line of the present invention via stimulation or suppression with compounds being tested can be done using immunoassay techniques and commercially available immunoassay antibodies, reagent and kit for mouse proteins.

Any type of immunoassay format may be used, including, without limitation, enzyme immunoassays (EIA, ELISA), radioimmunoassay (RIA), fluoroimmunoassay (FIA), chemiluminescent immunoassay (CLIA), counting immunoassay (CIA), immunohistochemistry (IHC), agglutination, nephelometry, turbidimetry or Western Blot. These and other types of immunoassays are well-known and are described in the literature, for example, in Immunochemistry, Van Oss and Van Regenmortel (Eds), CRC Press, 1994; and The Immunoassay Handbook, D. Wild (Ed.), Elsevier Ltd., 2005; and the references disclosed therein.

A preferred assay format for the present invention is the enzyme-linked immunosorbent assay (ELISA) format. ELISA is a highly sensitive technique for detecting and measuring antigens or antibodies in a solution in which the solution is run over a surface to which immobilized antibodies specific to the substance have been attached, and if the substance is present, it will bind to the antibody layer, and its presence is verified and visualized with an application of antibodies that have been tagged or labeled so as to permit detection. ELISAs combine the high specificity of antibodies with the high sensitivity of enzyme assays by using antibodies or antigens coupled to an easily assayed enzyme that possesses a high turnover number such as alkaline phosphatase (AP) or horseradish peroxidase (HRP), and are very useful tools both for determining antibody concentrations (antibody titer) in sera as well as for detecting the presence of antigen.

There are many different types of ELISAs; the most common types include “direct ELISA,” “indirect ELISA,” “sandwich ELISA” and cell-based ELISA (C-ELISA). Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized the detection antibody is added, forming a complex with the antigen. The detection antibody can be covalently linked to an enzyme, or can itself be detected by a secondary antibody which is linked to an enzyme through bioconjugation. Between each step the plate typically is washed with a mild detergent solution to remove any proteins or antibodies that are not specifically bound. After the final wash step the plate is developed by adding an enzymatic substrate tagged with a detectable label to produce a visible signal, which indicates the quantity of antigen in the sample.

In a typical microtiter plate sandwich immunoassay, an antibody (“capture antibody”) is adsorbed or immobilized onto a substrate, such as a microtiter plate. Monoclonal antibodies are preferred as capture antibodies due to their greater specificity, but polyclonal antibodies also may be used. When the test sample is added to the plate, the antibody on the plate will bind the target antigen from the sample, and retain it in the plate. When a second antibody (“detection antibody”) or antibody pair is added in the next step, it also binds to the target antigen (already bound to the monoclonal antibody on the plate), thereby forming an antigen ‘sandwich’ between the two different antibodies.

This binding reaction can then be measured by radio-isotopes, as in a radio-immunoassay format (RIA); by enzymes, as in an enzyme immunoassay format (EIA or ELISA); or other detectable label, attached to the detection antibody. The label generates a color signal proportional to the amount of target antigen present in the original sample added to the plate. Depending on the immunoassay format, the degree of color can be detected and measured with the naked eye (as with a home pregnancy test), a scintillation counter (for an RIA), or with a spectrophotometric plate reader (for an EIA or ELISA).

The assay then is carried out according to the following general steps:

Step 1: Capture antibodies are adsorbed onto the well of a plastic microtiter plate (no sample added);

Step 2: A test sample (such as human serum) is added to the well of the plate, under conditions sufficient to permit binding of the target antigen to the capture antibody already bound to the plate, thereby retaining the antigen in the well;

Step 3: Binding of a detection antibody or antibody pair (with enzyme or other detectable moiety attached) to the target antigen (already bound to the capture antibody on the plate), thereby forming an antigen “sandwich” between the two different antibodies. The detectable label on the detection antibodies will generate a color signal proportional to the amount of target antigen present in the original sample added to the plate.

In an alternative embodiment, sometimes referred to as an antigen-down immunoassay, the analyte (rather than an antibody) is coated onto a substrate, such as a microtiter plate, and used to bind antibodies found in a sample. When the sample is added, the antigen on the plate is bound by antibodies from the sample, which are then retained in the well. A species-specific antibody (anti-mouse IgE for example) labeled with an enzyme such as horse radish peroxidase (HRP) is added next, which, binds to the antibody bound to the antigen on the plate. The higher the signal, the more antibodies there are in the sample.

In another embodiment, an immunoassay may be structured in a competitive inhibition format. Competitive inhibition assays are often used to measure small analytes because competitive inhibition assays only require the binding of one antibody rather than two as is used in standard ELISA formats. In a sequential competitive inhibition assay, the sample and conjugated analyte are added in steps similar to a sandwich assay, while in a classic competitive inhibition assay, these reagents are incubated together at the same time.

In a typical sequential competitive inhibition assay format, a capture antibody is coated onto a substrate, such as a microtiter plate. When the sample is added, the capture antibody captures free analyte out of the sample. In the next step, a known amount of analyte labeled with a detectable label, such as an enzyme or enzyme substrate, added. The labeled analyte also attempts to bind to the capture antibody adsorbed onto the plate, however, the labeled analyte is inhibited from binding to the capture antibody by the presence of previously bound analyte from the sample. This means that the labeled analyte will not be bound by the monoclonal on the plate if the monoclonal has already bound unlabeled analyte from the sample. The amount of unlabeled analyte in the sample is inversely proportional to the signal generated by the labeled analyte. The lower the signal, the more unlabeled analyte there is in the sample. A standard curve can be constructed using serial dilutions of an unlabeled analyte standard. Subsequent sample values can then be read off the standard curve as is done in the sandwich ELISA formats. The classic competitive inhibition assay format requires the simultaneous addition of labeled (conjugated analyte) and unlabeled analyte (from the sample). Both labeled and unlabeled analyte then compete simultaneously for the binding site on the monoclonal capture antibody on the plate. Like the sequential competitive inhibition format, the colored signal is inversely proportional to the concentration of unlabeled target analyte in the sample. Detection of labeled analyte can be read on a microtiter plate reader.

In addition to microtiter plates, immunoassays are also may be configured as rapid tests. Like microtiter plate assays, rapid tests use antibodies to react with antigens and can be developed as sandwich formats, competitive inhibition formats, and antigen-down formats. With a rapid test, the antibody and antigen reagents are bound to porous membranes, which react with positive samples while channeling excess fluids to a non-reactive part of the membrane. Rapid immunoassays commonly come in two configurations: a lateral flow test where the sample is simply placed in a well and the results are read immediately; and a flow through system, which requires placing the sample in a well, washing the well, and then finally adding an analyte-detectable label conjugate and the result is read after a few minutes. One sample is tested per strip or cassette. Rapid tests are faster than microtiter plate assays, require little sample processing, are often cheaper, and generate yes/no answers without using an instrument. However, rapid immunoassays are not as sensitive as plate-based immunoassays, nor can they be used to accurately quantitate an analyte.

Where microtiter plates or strips are used, the capture antibody is immobilized within the wells. Techniques for coating and/or immobilizing proteins to solid phase substrates are known in the art, and can be achieved, for example, by a physical adsorption method, a covalent bonding method, an ionic bonding method, or a combination thereof. See, e.g., W. Luttmann et al., Immunology, Ch. 4.3.1 (pp. 92-94), Elsevier, Inc. (2006) and the references cited therein. For example, when the binding substance is avidin or streptavidin, a solid phase to which biotin was bound can be used to fix avidin or streptavidin to the solid phase. The amounts of the capture antibody, the detection antibody and the solid phase to be used can also be suitably established depending on the antigen to be measured, the antibody to be used, and the type of the solid phase or the like. Protocols for coating microtiter plates with capture antibodies, including tools and methods for calculating the quantity of capture antibody, are described for example, on the websites for Immunochemistry Technologies, LLC (Bloomington, Minn.) and Meso Scale Diagnostics, LLC (Gaithersburg, Md.).

In a preferred embodiment, the bioassay is a sandwich immunoassay. Preferably, the sandwich immunoassay of the present invention comprises the step of measuring the labeled secondary antibody, which is bound to the detection antibody, after formation of the capture antibody-antigen-detection antibody complex on the solid phase. The method of measuring the labeling substance can be appropriately selected depending on the type of the labeling substance. For example, when the labeling substance is a radioisotope, a method of measuring radioactivity by using a conventionally known apparatus such as a scintillation counter can be used. When the labeling substance is a fluorescent substance, a method of measuring fluorescence by using a conventionally known apparatus such as a luminometer can be used.

The sandwich immunoassay of the present invention may comprise one or more washing steps. By washing, the unreacted reagents can be removed. For example, when the solid phase comprises a strip of microliter wells, a washing substance or buffer is contacted with the wells after each step. Examples of the washing substance that can be used include 2-[N-morpholino]ethanesulfonate buffer (MES), or phosphate buffered saline (PBS), etc. The pH of the buffer is preferably from about pH 6.0 to about pH 10.0. The buffer may contain a detergent or surfactant, such as Tween 20.

The sandwich immunoassay can be carried out under typical conditions for immunoassays. The typical conditions for immunoassays comprise those conditions under which the pH is about 6.0 to 10.0 and the temperature is about 30 to 45° C. The pH can be regulated with a buffer, such as phosphate buffered saline (PBS), a triethanolamine hydrochloride buffer (TEA), a Tris-HCl buffer or the like. The buffer may contain components used in usual immunoassays, such as a surfactant, a preservative and serum proteins. The time of contacting the respective components in each of the respective steps can be suitably established depending on the antigen to be measured, the antibody to be used, and the type of the solid phase or the like.

In an alternative embodiment, competitive ELISA assays can be used to screen for candidate compounds that modulate TLR activity. In one aspect, a sandwich assay is employed wherein an innate binding partner, for example, a known ligand of the TLR, is coated on the bottom of ELISA plates comprising a commercially available multiwell format. Solubilized TLR or membrane preparations comprising TLR, which may be obtained by preparing a lysate of the present cells, can then be added to the wells of the plates. After a washing step, the test compound can be added at one or more concentrations to the wells containing the innate binding partner and TLRs. The mixture can be incubated for a sufficient period of time to allow the competition between the innate binding partner and the test compound for the TLR to reach equilibrium. The mixture can then removed from the wells using a gentle wash and any residual TLR is measured using an antibody directed to TLR, for example, Toll-like receptor 4 antibody (cat. No. 2219) from Cell Signaling Technology (Danvers, Mass.). If the presence of antibody labeled-TLR is lower in the assay wells containing the innate binding partner and test compound as compared to the innate binding partner in the absence of the test compound, then the test compound is a candidate TLR agonist or antagonist compound.

In some embodiments, cells of the present invention can be bound to the surface of a substrate and then incubated with a labeled innate binding partner for a TLR. Displacement of the labeled innate binding partner from the TLR with a test compound indicates that the test compound is a candidate TLR agonist or antagonist compound.

In another embodiment, assays contemplated by the present invention can be designed to screen for test compounds which can bind and modulate the activity of a TLR including, TLR 2 and/or TLR4, with its cognate secondary messengers and signal transduction partners. For example, triggering of the TLR pathway leads to the activation of NF-κB and subsequent regulation of immune and inflammatory genes. Upon activation, TLRs associate with a number of cytoplasmic adaptor proteins containing TIR domains including MyD88 (myeloid differentiation factor), MAL/TIRAP (MyD88-adaptor-like/TIR-associated protein), TRIF (Toll-receptor-associated activator of interferon) and TRAM (Toll-receptor-associated molecule). This association leads to the recruitment and activation of IRAK1 and IRAK4, which form a complex with TRAF6 to activate TAK1 and IKK. Activation of IKK leads to the degradation of IκB that normally maintains NF-κB inactivity by sequestering it in the cytoplasm. The activity of the compound can be determined by determining its effect on the production or suppression of one or more of the proteins involved in TLR signal transduction. Anti-TLR antibodies as well as antibodies specific for many of the proteins in the TLR signaling pathway are commercially available, for example, from Abeam (Cambridge, Mass.), Cell Signaling Technology, Inc. (Danvers, Mass.), Santa Cruz Biotechnology (Santa Cruz, Calif.), and others. For example, antibodies specific for MyD88, MAL/TIRAP, TRIF, TRAM, IRAK1, IRAK4, TAK1, IKK and NF-κB are commercially available, for example, from Cell Signaling Technology (Danvers, Mass.) or Santa Cruz Biotechnology (Santa Cruz, Calif.).

The antibodies used in the assays can be an anti-TLR antibody, such as anti-TLR4 or anti-TLR2, or an antibody specific for a protein involved in TLR signal transduction, including, for example, TRIF, MyD88, MDA-5, TRAF6, IRAK1, IRAK2, IRAK 3 and IRAK4 MAL/TIRAP, TRAM, TAK1, IKK and NF-κB. The antibodies may be used as capture or detection antibodies, depending on the assay format. The detection antibody may be directly conjugated with a detectable label, or an enzyme. If the detection antibody is not conjugated with a detectable label or an enzyme, then a labeled secondary antibody that specifically binds to the detection antibody is included. Such detection antibody “pairs” are commercially available, for example, from Cell Signaling Technology, Inc. (Danvers, Mass.)

Techniques for labeling antibodies with detectable labels are well-established in the art. As used herein, the term “detectable label” refers to a composition detectable by spectroscopic, photochemical, biochemical, immunochemical, or chemical means. The detectable label can be selected, e.g., from a group consisting of radioisotopes, fluorescent compounds, chemiluminescent compounds, enzymes, and enzyme co-factors, or any other labels known in the art. See, e.g., Zola, Monoclonal Antibodies: A Manual of Techniques, pp. 147-158 (CRC Press, Inc. 1987). A detectable label can be attached to the subject antibodies and is selected so as to meet the needs of various uses of the method which are often dictated by the availability of assay equipment and compatible immunoassay procedures. Appropriate labels include, without limitation, radionuclides, enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Evidot® quantum dots supplied by Evident Technologies, Troy, N.Y., or Qdot™ nanoparticles supplied by the Quantum Dot Corporation, Palo Alto, Calif.).

When the labeling substance is an enzyme, a method of measuring luminescence or coloration by reacting an enzyme substrate with the enzyme can be used. The substrate that can be used for the enzyme includes a conventionally known luminescent substrate, calorimetric substrate, or the like. When an alkaline phosphatase is used as the enzyme, its substrate includes chemilumigenic substrates such as CDP-star® (4-chloro-3-(methoxyspiro(1,2-dioxetane-3,2′-(5′-chloro)tricyclo[3.3.1.1.-sup.3.7]decane)-4-yl)disodium phenylphosphate) and CSPD® (3-(4-methoxyspiro(1,2-dioxetane-3,2-(5′-chloro)tricyclo[3.3.1.1.sup.3.7]-decane)-4-yl)disodium phenylphosphate) and colorimetric substrates such as p-nitrophenyl phosphate, 5-bromo-4-chloro-3-indolyl-phosphoric acid (BCIP), 4-nitro blue tetrazolium chloride (NBT), and iodonitro tetrazolium (INT). These luminescent or calorimetric substrates can be detected by a conventionally known spectrophotometer, luminometer, or the like.

In a currently preferred embodiment, the detectable labels comprise quantum dots (e.g., Evidot® quantum dots supplied by Evident Technologies, Troy, N.Y., or Qdot™ nanoparticles supplied by the Quantum Dot Corporation, Palo Alto, Calif.). Techniques for labeling proteins, including antibodies, with quantum dots are known. See, e.g., Goldman et al., Phys. Stat. Sol., 229(1): 407-414 (2002); Zdobnova et al., J. Biomed. Opt., 14(2):021004 (2009); Lao et al., JACS, 128(46):14756-14757 (2006); Mattoussi et al., JACS, 122(49):12142-12150 (2000); and Mason et al., Methods in Molecular Biology: NanoBiotechnology Protocols, 303:35-50 (Springer Protocols, 2005). Quantum-dot antibody labeling kits are commercially available, e.g., from Invitrogen (Carlsbad, Calif.) and Millipore (Billerica, Mass.).

Screening assays utilizing the cells of the present invention also may comprise a radiolabeled binding assay. The radio labeled binding assay, and variations thereof, can be designed using membrane preparations of TLR and thereby incubating the TLR with one or more test compounds in an assay mixture and for sufficient time for the test compound to specifically bind to the TLR. In some embodiments, a radiolabeled competitor (an innate binding partner) which is known to specifically bind to the binding domain of the TLR is added. If the test compound can displace any amount of the radiolabeled binding partner, then the test compound is said to specifically bind to the TLR, and is a candidate compound for further screening. In various embodiments, the innate binding partner can be labeled with any molecule, for example, radioisotope, fluorescent dyes, enzymatic reporters such as alkaline phosphatase, or horseradish peroxidase, biotin, avidin, enzyme or detection molecule, for example, HIS tag, FLAG tag and the like.

In another embodiment, whole cells of the present invention, or cell lysates prepared by lysing the cells, which express functional TLR 2 and/or TLR4, can be incubated with a test compound, and in a separate reaction with a known binding partner for TLR2 and/or TLR4, such as bacterial cell-surface lipopolysaccharides (LPS) heat shock proteins, fibrinogen, heparin sulfate fragments, etc. TLR ligands are commercially available from, for example, Imgenex (San Diego, Calif.) and InvivoGen (San Diego, Calif.). The samples are treated under the same assay conditions. The degree of TLR activation in the presence of the test compound and of the innate binding partner can be determined by measuring the activity of the TLR, e.g., by measuring the production or suppression of one or more of the proteins involved in TLR signal transduction, such as one or more of the proteins identified above. The presence and levels of these proteins can be determined, for example, by incubating the cells with labelled antibodies specific for one or more of the proteins, and measuring the amount of label in the cells with and without the test compound. Test compounds that can modulate the activity of one or more proteins involved in TLR signal transduction via interaction and specific binding with the TLR, including TLR2 and TLR4, as compared to a control solution having no test compound are TLR agonist or antagonist candidates.

TLR activation and modulation can also be assayed using test compounds in a cell-free system. Such screening methods can comprise the steps of providing membrane preparations comprising TLRs isolated from cells of the present invention. The membranes contain functional TLRs, and can be placed into assay reactions comprising secondary messengers and transduction proteins, e.g., using labeled antibodies as described above. Test compounds that can increase or decrease the activity of proteins in the signal transduction pathway as a result of TLR modulation when the TLR is in contact with a test compound as compared to control assays in which the test compound is absent are TLR agonist or antagonist candidates.

Screening assays can be performed to determine whether a test compound can alter the structural morphology of the present alveolar macrophage cells or lung epithelial cells in cell culture. Alveolar macrophage cells or lung epithelial cells derived from the present immorto-double mutant mouse as described in Example 3 can be incubated in the presence and absence of a test compound to determine whether structural changes can be observed, e.g., by light microscopy. After a time, the cells incubated with the test compound are examined microscopically to determine the presence of morphological differences compared to control cells of the same type incubated under the same conditions without the test compound. The presence of morphological changes, such as a decrease in the symptoms or progression of fibrosis, in cells treated with the test compound indicates that the compound may show promise for ameliorating or treating HPS, lung fibrosis or inflammation.

In one embodiment, a method for screening for a candidate compound that modulates the activity of a TLR receptor for the treatment of HPS, lung fibrosis, inflammation, or a related condition comprises: (a) contacting an alveolar macrophage or lung epithelial cell of the present invention with a first sample comprising a test compound, thereby forming a treated cell, and thereafter observing one or more phenotypic parameters thereof selected from the group consisting of cell size, cell proliferation, and cell morphology and combinations thereof; (b) contacting under the same conditions as used in (a), a second sample identical in composition to the first sample minus the test compound, thereby forming a control cell, and thereafter observing one or more phenotypic parameters thereof selected from the group consisting of cell size, cell proliferation, and cell morphology and combinations thereof; (c) comparing the observed phenotypic parameters to find a significant difference between said treated and control cells; (d) verifying that the test compound is a ligand for a TLR receptor; and (e) identifying, based on a significant difference found in (c), the test compound as a candidate compound that modulates the activity of the TLR receptor for the treatment of HPS, lung fibrosis, inflammation, or a related condition.

Other types of bioassays may be used to determine the effect of a test compound on the activity of a TLR. These assays include, but are not limited to, immunological assays, including Western blots, immunohistochemistry, solid-phase radioimmunoassays, in situ hybridizations, and immunoprecipitations. Antibodies various TLRs as well as to many of the proteins involved in signal transduction from TLRs, are known in the art, and are commercially available or can be readily generated using well-known techniques. These same factors can be detected by detecting their cognate nucleic acid including DNA and mRNA. In some embodiments, these assays include, but are not limited to, in situ hybridization, Reverse Transcript-Polymerase Chain Reaction (RT-PCR), quantitative RT-PCR and Northern blotting. Antibodies against various TLRs and related pathway proteins and reagents and kits for using them in Western or Northern blot assays are commercially available, for example, from Cell Signaling Technology (Danvers, Mass.).

In a method for screening for candidate compounds that activate or inhibit TLRs, especially TLR2 and/or TLR4, alveolar macrophage cells or lung epithelial cells prepared as described in Example 3, are grown or incubated in medium containing a test compound. The reference to which the test compound is compared can be a standard or control of any type, including average data generated in other assays, data generated in a control sample run concurrently with a given test. The presence, concentration, or amount of one or more proteins involved in signal transduction from TLRs in the cell is determined using a protein detection assay as described above. Test compounds that result in an increase in the treated cells in the amount of one or more proteins involved in signal transduction from TLRs compared to cells grown or incubated under similar conditions but without the test compound show promise as TLR agonists. Conversely, test compounds that result in a decrease in the treated cells in the amount of one or more proteins involved in signal transduction from TLRs compared to cells grown or incubated under similar conditions but without the test compound show promise as TLR antagonists.

Examples of detectable labels that allow for the detection of antibodies include fluorescent molecules (for example, fluorescein, rhodamine, Hoechst 33258, or Texas red), enzymes (for example, horseradish peroxidase, alkaline phosphatase, or beta-galactosidase), gold particles, radioactive isotope, and biotin. An assay format is selected based on the labeling moiety used. For example, fluorescence microscopy can be used to detect fluorescently labeled antibodies. For cells stained with enzyme-conjugated antibodies, the cells are further treated with an appropriate substrate for conversion by the antibody-bound enzyme, followed by examination by light microscopy. Gold-particle labeled antibodies can be detected using light or electron microscopy. Isotope-labeled antibodies can be detected using radiation-sensitive film.

For cells stained with biotin-conjugated antibodies, the cells are further treated with streptavidin or avidin. The streptavidin or avidin is conjugated to a moiety that allows for detection such as, for example, a fluorescent molecule, an enzyme, gold particles, or radioactive isotope. In some embodiments, the cells are co-stained with an antibody or antibodies specific for particular subcellular compartments (e.g., nucleus, cytoplasm, endoplasmic reticulum, etc.). Using any one of these techniques, or any other known technique for detecting antibodies in antibody-stained cells, the subcellular distribution of protein factors that are implicated in the etiology and/or progression of HPS, lung fibrosis, inflammation or related condition can be determined.

The invention is further illustrated by the following non-limiting examples.

### EXAMPLES

### Example 1

### Production of Immortalized HPS Double Mutant Mice

Materials and Methods

Mice containing the SV40tsA58 transgene were purchased commercially (Immortomouse® Homozygous, Charles River Laboratories, Wilmington, Mass.). HPS double mutant mice C57/BL6-HPS1ep Apb1pe, which are double homozygous for the pale ear (HPS1ep) and pearl (Apb1pe) genes (referred to hereinafter as the “ep/pe double mutant”), are described in Lyerla et al., Am J Physiol Lung Cell Mol Physiol, 285:L643-L653 (2003). The immortomice were mated with the ep/pe double mutant mice to produce an F1 hybrid generation. Genetically, this F1 generation is heterozygous for the three coat color genes −b+/b; pe+/pe; ep+/ep− and for the SV40tsA58 marker (sv+/−). These F1 mice have the coat color of the immortomouse strain, and all will test positive for the presence of the SV40tsA58 transgene if the immortomouse animals are homozygous for this gene.

The presence of the SV40tsA58 transgene was tested using PCR from DNA of tail tip samples. Samples are taken from three-week old F1 mice, as well as from immortomice and HPS ep/pe parental double mutants as controls, and extracted using DirectPCR (Tail) lysis reagent (Viagen Biotech Inc., Los Angeles, Calif.) according to the manufacturer's instructions. Briefly, tail tip samples were lysed overnight with rotation in a microcentrifuge tube at 55° C. in 200 to 300 μL of DirectPCR Lysis reagent (Viagen Biotech Inc.) with 0.5 mg/mL proteinase K (Sigma Chemical Co., St. Louis, Mo.). Samples then were raised to 85° C. in a water bath for 45 min and clarified by centrifugation in a microcentrifuge. Supernatants were stored at 4° C. for immediate use, or frozen at −20° C. for long-term storage.

This target DNA is used for PCR amplification. The PCR reaction mixture contained 5 μL Taq buffer, 1 μL dNTP, 0.2 μL Taq polymerase (all from New England BioLabs, Inc., Ipswich, Mass.), 2 μL primers (10 uM concentrations; (Dory et al., 2003; purchased from IDT, Coralville, Iowa), from 0.5 to 5 μL target DNA, and brought to 50 μL total reaction mixture with 38.8 μL water. Samples were amplified on a thermal cycler (MJ Research PTC-200) with 48-well dual heads and gradient or constant temperature heads, that is, “warm bonnets”, using the following cycling protocol: 2 minutes at 95° C., forty cycles of 0.5 minutes 95° C., 0.5 minutes 55° C., 1 minute 72° C. The reaction was stopped and held at 4° C. until samples were retrieved. Samples were separated by agarose gel electrophoresis (2% Sea-Kern Gold Agarose in TBE buffer containing ethidium bromide; Lonza, Rockland, Me.) for 1 to 2 hours at 290 V and 100 mA, and examined for products using UV illumination.

The size of the product from the SV40tsA58 primer set was 0.277 kb, the expected product size when the primers are mapped onto the SV40 genome. Also, the primer set amplified tail tip DNA extracts only from animals known or expected to bear the SV40tsA58 gene, so it exhibits specificity as well as appropriate product size.

The F1 mice, all heterozygous for the genes of interest, were backcrossed to the HPS ep/pe double mutant parental strain in order to produce an F2 generation. Sixteen different phenotypes in equal proportions ( 1/16th of each type among all offspring) must be recognized in the F2s: eight different coat color types, and each of these either sv+/−, or −/− for the SV40tsA58 transgene:

| Genotype | Phenotype |
|---|---|
| 1. b+/b; ep+/ep; pe+/pe; sv+/− | agouti with transgene (like C3H strain) |
| 2. b+/b; ep+/ep; pe+/pe; −/− | agouti without transgene |
| 3. b+/b; ep/ep; pe+/pe; sv+/− | agouti-pale ear with transgene |
| 4. b+/b; ep/ep; pe+/pe; −/− | agouti-pale ear without transgene |
| 5. b+/b; ep+/ep; pe/pe; sv+/− | agouti-pearl with transgene |
| 6. b+/b; ep+/ep; pe/pe; −/− | agouti-pearl without transgene |
| 7. b+/b; ep/ep; pe/pe; sv+/− | agouti-double mutant with transgene |
| 8. b+/b; ep/ep; pe/pe; −/− | agouti-double mutant without transgene |
| 9. b/b; ep+/ep; pe+/pe; sv+/− | black with transgene |
| 10. b/b; ep+/ep; pe+/pe; −/− | black without transgene |
| 11. b/b; ep/ep; pe+pe; sv+/− | black-pale ear with transgene |
| 12. b/b; ep/ep; pe+/pe; −/− | black-pale ear without transgene |
| 13. b/b; ep+/ep; pe/pe; sv+/− | black-pearl with transgene |
| 14. b/b; ep+/ep; pe/pe; −/− | black-pearl without transgene |
| 15. b/b; ep/ep; pe/pe; sv+/− | black-double mutant with transgene |
| 16. b/b; ep/ep; pe/pe; −/− | black-double mutant without transgene |
| | (like HPS ep/pe strain) |

Only one-eighth of the F2 offspring were immortoHPS double mutant mice (#7, the agouti double mutant animals with the transgene, plus #15, the black double mutant animals with the transgene. These two strains are used for developing immortalized cell lines and for further crosses to maintain these genotypes.

Histological Analyses of ImmortoHPS Double Mutant Mice.

About ⅛th of the new HPS double mutant strain carries C3H genes. These mice were tested for abnormal ATII cells, the most reliable cellular marker for the development of lung fibrosis in the highly inbred C57/BL6-HPS1ep A3pb1pe—J HPS double mutant strain. This was accomplished using histological methods for retrieval of alveolar macrophages by bronchial alveolar lavage (BAL), and flotation fixation of lungs for processing, staining, and immunohistochemical analyses.

Obtaining lung samples. For histological analyses and BAL sampling, mice were weighed using a triple beam balance (Ohaus Scale Corp., Florham Park, N.J.) to the nearest 0.5 gm, and anesthetized with tribromomethanol (Avertin) at 120 mg/kg body weight. Avertin was prepared by dissolving 25 g of the bromylated methanol in 15.5 ml T-amyl alcohol by constant stirring in the dark, and storing at room temperature as stock solution. This solution was diluted 1:80 with sterile physiological saline and stored at 4° C. without light exposure. After warming of the anesthetic to room temperature, animals were injected i.p. in the right posterior quadrant of the abdomen and considered fully anesthetized when they no longer exhibited a twitch reflex to pinches of the toe webbing. They were then placed on a surgery board in a supine position and fastened with the use of surgical thread attached to needles impaled on the board. After sterilization with 70% alcohol on the surface, the mice were opened at the abdominal region with the use of fine scissors and forceps, and exsanguinated by severing the left renal artery. Then the neck region was opened and the trachea exposed posterior to the larynx. After displacing the muscle surrounding the trachea and providing a small hole with sterilized fine-tipped scissors, it was cannulated using a sterile blunt 18 gauge (or 23 gauge for mice of one month old) syringe needle (Lure-Lok Hub, Becton, Dickinson & Company, Rutherford, N.J.) which was tied to the trachea with the use of 3-0 black braided silk suture (Genzyme, Fall River, Mass.) to prevent leakage. The chest was opened with the use of forceps and scissors, and the diaphragm cut to expose the lung. The whole lung was lavaged using 1.0 ml PBS 3 times (or 0.6 ml 5 times for mice younger than two months). The lung was then fully inflated and fixed by 10% buffered formalin for histological processing.

Histological preparations. Sections of 5 μm thickness in paraffin blocks were prepared from formalin fixed lungs using standard methods. The lungs were soaked in 10% (v/v) buffered formalin for over 24 hr but less than 1 week, separated from the trachea and other attached tissues, and dehydrated in 50%, 70%, 95% and 95% (v/v) alcohol concentrations sequentially, over 1 hr for each treatment. They then were moved to absolute alcohol for 1.5 hr twice for final dehydration. Tissue clearance was done by two treatments in xylene, 1 hr each time. The cleared tissues were moved to freshly melted paraffin at 56° C. for 30 min, 1.5 hr and 1 hr serially. A mold was filled with fresh paraffin which was allowed to stand at room temperature until the paraffin at the bottom of the mold was partially solidified. The tissues were placed into the bottom of the mold with the use of forceps. The mold was sealed by the block frame (Tissue-Tek, Miles Inc, Elkhart, Ind.) and filled with paraffin. The finished preparations were cooled to 4° C. by refrigeration overnight and the molds and blocks then separated. The blocks were sectioned at 5 μm using a Spencer 820 microtome (Spencer Scientific Corporation, Derry, N.H.). The sections were mounted on positively charged slides (VWR International, West Chester, Pa.) and baked at 56° C. for 30 min.

Prior to staining using the Masson trichrome method, the slides were deparaffinized and rehydrated by steeping 5 min sequentially in xylene, xylene, 100% alcohol, 100% alcohol, 95% alcohol, 95% alcohol, 70% alcohol, 50% alcohol and deionized water. Slides were used immediately for subsequent staining or stored in deionized water for 1 to 3 hours prior to staining. The deparaffinized and rehydrated slides were treated with Bouin's fixative (150 ml saturated picric acid, 50 ml formalin, and 10 ml glacial acetic acid as the working solution) for more than 1 but less than 3 hr at 56° C. The cooled slides were washed thoroughly by deionized water. When there was no yellow color left on the slides, they were transferred to Weigert's hematoxylin (freshly made working solution of 1 part A and 1 part B, where A=5.0 g Hematoxylin in 500.0 ml 95% alcohol and B=20.0 ml 29% (w/v) ferric chloride, 5.0 ml hydrochloric acid and 475.0 ml deionized water), incubated for 20 min, and rinsed in deionized water. Then the slides were transferred sequentially to Biebrich scarlet-acid fuchsin solution (2.7 g Biebrich scarlet, 0.3 g acid fuchsin, 3 ml glacial acetic acid in 300 ml deionized water) for about 20 min, phosphomolybdic/phosphotungstic acid solution (25 g of each acid in 1000 ml deionized water) for about 15 min to remove the Biebrich's scarlet out of collagen but not out of the cytoplasm, aniline blue (2.5 g with 1 ml glacial acetic acid in 100 ml deionized water) for about 15 min, a deionized water rinse, and freshly made 1% acetic acid solution for about 1 min. The times of the treatments were adjusted according to the development of the staining reactions. A commercially provided Masson trichrome stain kit (Richard-Allan Scientific, Kalamazoo, Mich.) was also used, with no apparent differences in staining quality.

Immunohistochemistry (IHC). IHC single staining was done either manually or by machine. For manual preparations, paraffin sections on slides were hydrated by three 5 min rinses in xylene, and 5 min treatments of decreasing alcohol concentrations: 100, 100, 95, 70, 50%), followed by two treatments in distilled water. Antigen retrieval was done in 10 mM citrate buffer (1.92 g citric acid in 1 L water, pH adjusted to 6.0 with 1 M NaOH) using a pressure cooker in a microwave oven, 3 cycles at 5 min each, 10% power, with half the citrate buffer replaced and cooling between each treatment. Slides were then brought to room temperature for 15 min in a water bath. They were blotted to remove excess water, soaked in phosphate buffered saline (PBS) for 5 min, and treated for 30 min in H₂O₂ (10 mL of 0.3% H₂O₂ in 40 mL methanol). The antibody used was raised in goats, so slides were pretreated for non-specific reaction with the antibody using freshly prepared 2.5% goat serum for 20 min. Sections were then incubated overnight at 4° C. in 1:50 dilution primary anti-surfactant C antibody. They were washed 3 times for 5 min each in Tween/PBS (0.1% Tween 20 in PBS), drained, and incubated with biotin-labeled secondary antibody (1:50 dilution) for 30 min. Fully treated slides were then placed into avidin-conjugated horseradish peroxidase with diaminobenzidine substrate in the dark until desired staining, rinsed in tap water, counterstained with hematoxylin, blued, and dehydrated for mounting in xylene soluble mounting fluid.

For machine staining, a BenchMark® XT IHC/ISH staining apparatus (Ventana Medical Systems, Inc, Tucson, Ariz.), using the manufacturer's detection kits with manual adjustments was used. The apparatus automates the classical procedures of IHC including deparaffinization, rehydration, antigen retrieval, any pretreatments such as endogenous peroxidase inactivation and protein blocks, primary antibody and secondary antibody treatments, chromogen development and counterstaining. For horse radish peroxidase/diaminobenzoic acid (HRP/DAB) staining, the essential pretreatment after antigen retrieval and before primary antibody treatments was endogenous peroxidase inactivation. The chromogen development included HRP-linked avidin reaction and HRP/DAB reaction, because the manufacturer's DAB detection kit uses biotin-labeled secondary antibody to amplify the color.

For all the kit programs, the time for antigen retrieval, primary antibody treatment, secondary antibody treatment and counterstaining can be modified. Optional steps can be added or deleted within the provided programs. Antibodies can be applied automatically or manually according to user's needs. The reagents and slides placed on heating pads are recognized by company-assigned or user-printed barcodes. Because of the automations provided by this apparatus, such as heating systems, liquid coverslip, and mixing stations, the efficiency of IHC is greatly improved over manual methods. Usually 30 slides can be processed within a 5 hr time frame, for example, which may take up to 2 days or more using non-automated methods. HRP/DAB was accomplished with the use of XT iVIEW DAB Open Kit. Freshly prepared reagents are listed below (Table 1).

TABLE 1
Freshly prepared reagents for HRP/DAB IHC.

| Reagents | | Dilution |
|---|---|---|
| Goat serum stock solution (Jackson Immunoresearch, | | 1:40 in |
| West Grove, PA) dissolved in 10 ml sterilized deionized | | sterilized |
| water | | deionized water |
| Rabbit | Anti-TGF β1 (Santa Cruz Biotech., Santa | 1:200 in 2.5% |
| anti- | Cruz, CA.) | goat serum |
| mouse | Anti-prosurfactant C (Fitzgerald Industries | 1:3000 in 2.5% |
| primary | Intl. 34 Junction Square Drive, Concord MA | goat serum |
| IgG | 01742) | |
| Goat anti- | Biotinylated goat anti-rabbit secondary | 1:500 in 2.5% |
| rabbit 2nd | antibody (Jackson Immunoresearch, West | goat serum |
| IgG | Grove, PA) | |

Two steps were added to eliminate nonspecific staining in HRP/DAB IHC: first, a 2.5% (v/v) goat serum treatment was added before primary antibody treatment and after endogenous peroxidase inactivation to block the highly charged collagen and connective tissue elements which can attach to antibodies; second, an endogenous biotin block reaction was added after the primary antibody treatment and before the secondary antibody treatment in order to avoid nonspecific avidin attachment. In this HRP/DAB procedure, the deparaffinized and rehydrated slides were set to be blocked in 2.5% goat serum for 16 min, treated with a rabbit anti-mouse primary antibody for 16 min, with biotinylated goat anti-rabbit secondary antibody for 16 min (Table 1), and counterstained by hematoxylin for 4 min followed by bluing agent (saturated Li₂CO₃ solution) for 4 min. The prosurfactant C antibody is specific for alveolar type II cells, the affected cells in the HPS double mutant mouse (Lyerla et al., 2003 supra).

### Example 2

### Cell Cultures from Bronchial Alveolar Lavage Samples

BAL samples were centrifuged at 4° C. with a super-speed centrifuge (Sorvall RC-6, Thermo Scientific, Rockford, Ill.), and the cell-free supernatant stored at −80° C. for further studies. The cell pellets were treated with 5 ml of sterile red blood lysis buffer (8.3 g NH₄Cl, 1.0 g KHCO₃ and 1.8 ml of 5% (w/v) EDTA dissolved in 1000 ml of deionized water) for 5 min. The cells were washed by PBS, resuspended in RPMI culture medium (Lonza) supplemented with 10% (v/v) fetal calf serum (Atlanta Biologicals, Atlanta, Ga.), 2 mM glutamine, and penicillin/streptomycin (Lonza). Cells collected as a single BAL sample were cultured in a well of a 48-well plate at 33° C. and 5% CO₂ partial pressure atmosphere. They were observed periodically with the use of an inverted microscope (Olympus CK), medium changed weekly, and cells passaged with the use of 0.25% trypsin/EDTA (Lonza) for detaching attached cells. This requires rinsing of the cells with PBS, adding 0.5 mL trypsin for 1 min at room temperature, aspirating the trypsin solution to form a thin film on the cells, and incubating at 33° C. for 10 to 20 min. The cells were harvested in culture medium, and passaged into 2 wells for a split ratio of 1:2 per well. As wells began to become confluent with these slowly dividing cells; the entire well of cells was passaged into a larger vessel using the same method. When sufficient numbers of cells were obtained, a portion of them was frozen in liquid nitrogen for long term storage while the remainder was kept in culture as an established line for further study. This includes differentiation at 37° C., the temperature that blocks proliferation and causes the cells to undergo maturation to the differentiated state, and characterization of this state in order to identify the cell type resulting from this procedure.

Results:

The F1 animals from the HPS double mutant C57/BL6-HPS1ep Ap3b1pe—J X Immortomouse® cross are heterozygous for the coat color genes and appear phenotypically like the ep/pe parents, and also are heterozygous for the SV40tsA58 transgene. When these F1s were backcrossed to the HPS double mutant C57/BL6-HPS1ep Ap3b1pe—J animals, eight different coat color phenotypes were seen in equal proportions, and half of these carry the SV40tsA58 transgene. The HPS ep/pe double mutant animals in this F2 stock possess abnormal ATII cells, and those carrying the transgene provide cells from bronchial alveolar lavage samples that are capable of proliferating at 33° C. in RPMI culture medium.

### Example 3

### Cell Lines Derived from ImmortoHPS Double Mutant Mice

Two different lung cell lines, one from alveolar macrophages and the other from lung epithelium, were derived from eppe double mutant mice in the F2 generation that were positive for tsA58. The two lines have the expected differences in growth patterns and morphologies relevant to their separate origins. They also possess distinguishing differences in chromosome morphologies. The presence of the tsA58 transgene allows for ease of providing established cell lines from this strain with euploid chromosomes, and for one of these lines, the telocentric condition for the species is maintained. This contrasts with the effect of developing immortal cell lines by direct infection with SV40 transforming virus.

The immortoHPS animals were produced by crossing eppe double mutant males with Immortomouse® females that were homozygous for the tsA58 transgene as described in Example 1. All F1 animals were heterozygous for the pigment genes (pe/pe+; ep/ep+; a/a+) and carried the tsA58 transgene (tsA58/−) as determined by visual inspection for coat color phenotype and PCR for tsA58. F1 males and females were testcrossed with eppe mates to produce an F2 with eight different coat color phenotypes, each with ½ probability of carrying the tsA58 transgene.

The F2 HPS eppe double mutant animals, either black (a/a) or agouti (a+/a), were screened for the tsA58 transgene using PCR, and tsA58+ animals used for the production of two cell lines. All experimental work with the mice was approved by the IACUC and in accordance with the NIH Guide for Laboratory Animal Research. An alveolar macrophage line (WLS) was derived from bronchial alveolar lavage samples taken under sterile conditions and cultured initially in RPMI medium supplemented with 10% (v/v) fetal calf serum and antibiotics at 33° C. with 5% CO2 atmosphere in 48-well culture plates. Outgrowth of cells from a single well was subcultured by trypsinization in DMEM medium with the same supplements and culture conditions until sufficient numbers of cells were available for cell freezing and subculturing in T-75 flasks or PD100 culture vessels.

A lung epithelial line (1e3p2) was derived from lung tissue that had been digested in situ with dispase after first blanching the lung with saline and lavaging to remove free alveolar cells. The resulting cell suspension digest was panned in antibody coated plates to remove non-epithelial cells, and floating epithelial cells cultured in 48-well plates for attachment and formation of monolayers in DMEM culture medium at 33° C. and 5% CO₂. The cultures were expanded by detachment with trypsin and subcultured under the same conditions to provide sufficient numbers of cells for passaging in larger cell culture vessels and freezing samples for long term storage.

Both cell lines have been in continuous cultivation for over 50 passages (100+ cell doublings), and have maintained their separate growth and morphological characteristics over this period. Chromosome preparations were made of metaphase spreads collected after treatment of cultures with 0.1 ug/mL colcemid (Roche Applied Science, Indianapolis, Ind.) for 1½-2 hr using standard methods, and photomicrographs taken with a Spot RT Slider digital camera (Diagnostic Instruments, Inc., Sterling Heights, Mich.) attached to a Nikon E600 compound microscope.

Cell morphologies of the two lines were reflective of their separate origins. The WLS alveolar macrophage cells grow from colonies of individual cells with extended cytoplasmic processes and do not reach confluency, whereas the 1e3p2 lung epithelial cells form a monolayer of attached cells that can become 100% confluent.

The WLS line appears to be homogeneous with respect to its cell type, whereas the 1e3p2 line is heterogeneous and may be comprised of more than one cell type. The metaphase spreads from lung epithelial cells contain only telocentric chromosomes. The structures of the individual chromosomes are normal and do not exhibit abnormalities such as dicentric or double minute chromosomes that are often found in cell lines treated directly with the SV40 virus with the goal of making immortal lines. There may be greater than normal stability of the 1e3p2 lung epithelial line with continuous cultivation due to its essentially standard karyotype, except for higher levels of ploidy, for this organism.

### REFERENCES

1. Huizing et al., “Hermansky-Pudlak syndrome and Chediak-Higashi syndrome: disorders of vesicle formation and trafficking.” Thromb Haemost, 86:233-245 (2001).
2. Spritz, “Multi-organellar disorders of pigmentation: intracellular traffic jams in mammals, flies and yeast.” Trends Genet, 15:337-340 (1999)
3. Oh et al., “Positional cloning of a gene for Hermansky-Pudlak syndrome, a disorder of cytoplasmic organelles.” Nat Gen, 14:300-306 (1996).
4. Brantly et al., “Pulmonary function and high-resolution CT findings in patients with an inherited form of pulmonary fibrosis, Hermansky-Pudlak syndrome, due to mutations in HPS-1.” Chest, 117:129-136 (2000).
5. Nakatani et al., “Interstitial pneumonia in Hermansky-Pudlak syndrome: significance of florid foamy swelling/degeneration (giant lamellar body degeneration) of type-2 pheumocytes.” Virchows Arch, 437:304-313 (2000).
6. Feng et al., “Mouse pale ear (ep) is homologous to hunan Hermansky-Pudlak syndrome and contains a rare AT-AC intron.” Hum Mol Genet, 6:793-797 (1997).
7. Gardner et al., “The mouse pale ear (ep) mutation is the homolog of human Hermansky-Pudlak syndrome (HPS).” Proc Natl Acad Sci (USA), 94:9238-43 (1997).
8. Feng et al., “The β_{3A}-subunit gene (Ap3b1) of the AP-3 adaptor complex is altered in the mouse hypopigmentation mutant pearl, a model for Hermansky-Pudlak Syndrome and night blindness.” Hum Mol Genet, 8:323-330 (1999).
9. Suzuki et al. “The gene mutated in cocoa mice, carrying a defective organelle biogenesis, is a homolog of the human Hermansky-Pudlak syndrome-3 gene.” Genomics, 78:30-37 (2001).
10. Suzuki et al. “Hermansky-Pudlak syndrome is caused by mutations in HPS4, the human homolog of the mouse light-ear gene.” Nat Genet, 30:321-324 (2002).
11. Zhang et al., “Ruby-eye-2 and ruby-eye encode mouse orthologs of the genes mutated in human Hermansky-Pudlak syndrome types 5 and 6.” Nat Genet, 33:145-153 (2003).
12. Lyerla et al., “Aberrant lung structure, composition and function in a murine model of Hermansky-Pudlak syndrome.” Am J Physiol Lung Cell Mol Physiol, 285:L643-L653 (2003).
13. Young et al., “Lung-restricted macrophage activation in the Pearl mouse model of Hermansky-Pudlak syndrome.” J. Immunol., 176:4361-4368 (2006).
14. Petit et al., Virology, 127:74-82 (1983).
15. Land et al, Nature, 304:596-602 (1983).
16. Ruley, Nature, 304:602-606 (1983).
17. Jat and Sharp J. Virology, 59: 746-750 (1986).
18. Rassoulzadegan et al, Nature, 300:713-718 (1982).
19. Phelps et al, Cell 53: 539-547 (1988).
20. Burns et al., Br. J. Cancer, 59:755-60 (1989).
21. Bayley et al., Exp. Cell Res., 177:232-36 (1988).
22. Santerre et al., Proc. Nat'l Acad, Sci USA, 78:4339-43 (1981).
23. Jat et al., Molecular and Cellular Biology, 6:1204-1217 (1986).
24. Palmiter et al., Nature, 316:457-460 (1985).
25. U.S. Pat. Nos. 5,866,759 and 5, 688,692.
26. Feng et al., Hum Mol Genet., 6:793-97 (1997).
27. Cabanski et al., Am J. Respir. Cell Mol Biol., 38:26-31 (2008).

What is claimed is:

1. A mutant mouse comprising in each cell:
a. homozygous mutations in one, two, or more genes that result in a Hermansky-Pudlak Syndrome phenotype; and
b. a transgene encoding a temperature-sensitive protein that is inactive at physiological temperatures.

2. The mutant mouse of claim 1 wherein the genes are selected from the group consisting of: Hps1, Hps2, Hps3, Hps4, Hps 5, Hps6, Ap3b1 and Ap3d.

3. The mutant mouse of claim 2 wherein the genes comprise Hps1 and Ap3b1.

4. A mutant mouse obtained by crossing a mouse comprising homozygous mutations in at least two genes that result in a Hermansky-Pudlak Syndrome phenoptype; and a transgenic mouse comprising a transgene encoding a temperature-sensitive protein that is inactive at physiological temperatures.

5. The mutant mouse of claim 4 wherein the genes comprise Hps1 and Ap3b1.

6. A cell line obtained by isolating cells from the mutant mouse of claim 1.

7. The cell line of claim 6 where in the cells are bronchial alveolar or lung epithelium cells.

8. An isolated cell comprising:
a. homozygous mutations in at least two genes that result in a Hermansky-Pudlak Syndrome phenoptype; and
b. a transgene encoding a temperature sensitive protein that is inactive at physiological temperatures.

9. The isolated cell of claim 8 wherein the genes are selected from the group consisting of: Hps1, Hps2, Hps 3, Hps4, Hps 5, Hps6, Ap3b1 and Ap3d.

10. The isolated cell of claim 9 wherein the genes comprise Hps1 and Ap3b1.

11. An assay for determining a biological effect of an agent, comprising a cell line wherein each cell comprises:
a. homozygous mutations in at least two genes that result in a Hermansky-Pudlak Syndrome phenoptype; and
b. a transgene encoding a temperature sensitive protein that is inactive at physiological temperatures.
